(19) United States
(12) Patent Application Publication
(10) Pub. No.: US 2026/0290622 A1
(43) Pub. Date: Sep. 24, 2026

(54) ONLINE COLLABORATIVE MEDICAL PLATFORM FOR FACILITATING COLLABORATION BETWEEN REMOTE MEDICAL PRACTITIONERS

(71) Applicant: Cilag GmbH International, Zug (CH)

(72) Inventors: Shan Gowri Jegatheeswaran, Santa Clara, CA (US); Raymond Samuel Fryrear, II, Cincinnati, OH (US); Fiona Middlemiss Haig, York (GB); Tyler Lee Hartley, Seattle, WA (US); Alexandre Hennen, New York, NY (US); Vikram Mohan, San Francisco, CA (US); Christine Purcell, New York, NY (US); Jamie Gavin Roots, Madrid (ES)

(21) Appl. No.: 19/689,826

(22) Filed: May 27, 2026

Related U.S. Application Data
(63) Continuation of application No. PCT/IB2024/062179, filed on Dec. 4, 2024.
(60) Provisional application No. 63/605,879, filed on Dec. 4, 2023.
(60) Provisional application No. 63/641,754, filed on May 2, 2024.
(60) Provisional application No. 63/661,015, filed on Jun. 17, 2024.
(60) Provisional application No. 63/661,858, filed on Jun. 19, 2024.
(60) Provisional application No. 63/717,950, filed on Nov. 8, 2024.
(60) Provisional application No. 63/718,000, filed on Nov. 8, 2024.
(60) Provisional application No. 63/719,015, filed on Nov. 11, 2024.

(51) Int. Cl. G16H 80/00; G16H 50/70

(52) U.S. Cl. CPC G16H 80/00; G16H 50/70

(57) ABSTRACT

A collaborative medical platform facilitates remote collaboration relating to medical procedures during preprocedural, intraprocedural, and postprocedural stages of a medical case. In a preprocedural stage, the collaborative medical platform collects various data relevant to a medical procedure and enables various medical practitioners to exchange data to support preparation for the medical procedure. In the intraprocedural stage, the platform facilitates telepresence sessions to enable real-time collaboration during a procedure. During the postprocedural stage, the collaborative medical platform may generate various metrics relevant to evaluating performance. The collaborative medical platform may furthermore intelligently generate recommendation or other content based on the captured preprocedural, intraprocedural, and postprocedural data.

### CROSS-REFERENCE TO RELATED APPLICATIONS

This application is a continuation of PCT Application No. PCT/IB2024/062179 filed on Dec. 4, 2024, which claims the benefit of the following applications: U.S. Provisional Patent Application No. 63/605,879 filed on Dec. 4, 2023, U.S. Provisional Patent Application No. 63/641,754 filed on May 2, 2024, U.S. Provisional Patent Application No. 63/661,015 filed on Jun. 17, 2024, U.S. Provisional Patent Application No. 63/661,858 filed on Jun. 19, 2024, U.S. Provisional Patent Application No. 63/717,950 filed on Nov. 8, 2024, U.S. Provisional Patent Application No. 63/718,000 filed on Nov. 8, 2024, and U.S. Provisional Patent Application No. 63/719,015 filed on Nov. 11, 2024. The contents of each of the foregoing applications are incorporated by reference herein.

### BACKGROUND

### Technical Field

The described embodiments relate to a system and method for a collaborative medical platform that facilitates collaboration between medical practitioners in relation to medical procedures.

### Description of the Related Art

When preparing for, performing, or monitoring outcomes of various medical procedures, collaboration between different medical practitioners may increase efficiency of procedures and may improve patient results. However, in conventional medical environments, it may be challenging or inconvenient to identify and engage other medical practitioners with relevant expertise for collaboration. Additionally, sharing of patient health information using informal communications systems like text and email may implicate various data privacy and compliance issues.

### BRIEF DESCRIPTION OF THE DRAWINGS

FIG. 1 is an example embodiment of a computing environment for an electronically-assisted medical procedure.

FIG. 2 is a block diagram of an example architecture for a collaborative medical platform.

FIG. 3A shows a first view of an example practitioner dashboard associated with a collaborative medical platform.

FIG. 3B shows a second view of an example practitioner dashboard associated with a collaborative medical platform.

FIG. 4A shows a first view of an example embodiment of a case page associated with a medical case in the collaborative medical platform.

FIG. 4B shows a second view of an example embodiment of a case page associated with a medical case in the collaborative medical platform.

FIG. 5 is an example embodiment of a case sharing interface associated with sharing a medical case in the collaborative medical platform.

FIG. 6 is an example embodiment of a case dashboard associated with a set of cases in a collaborative medical platform.

FIG. 7 is an example telepresence interface associated with a collaborative medical platform.

FIG. 8 is another example of a telepresence interface associated with a collaborative medical platform.

FIG. 9 is an example analytics dashboard associated with a collaborative medical platform.

FIG. 10 is an example video interface associated with a collaborative medical platform.

FIG. 11 is an example presentation interface associated with a collaborative medical platform.

FIG. 12 is a flowchart of an example embodiment of a method for operating a collaborative medical platform.

### DETAILED DESCRIPTION

The Figures (FIGS.) and the following description describe certain embodiments by way of illustration only. One skilled in the art will readily recognize from the following description that alternative embodiments of the structures and methods illustrated herein may be employed without departing from the principles described herein. Reference will now be made to several embodiments, examples of which are illustrated in the accompanying figures. Wherever practicable, similar or like reference numbers may be used in the figures and may indicate similar or like functionality.

A collaborative medical platform facilitates exchange of data between remote medical practitioners in relation to medical cases during preprocedural, intraprocedural, and postprocedural stages. The collaborative medical platform stores or enables access to patient records, imaging data, video data, telemetry data from medical equipment, biometric sensor data from patients, and other medical information that may be obtained prior to medical procedures being performed, during medical procedures, or after medical procedures are performed. The collaborative medical platform includes various interfaces that enable collaboration between medical practitioners via shared media libraries, content feeds associated with specified cases, telepresence sessions recorded during procedures or simulations of procedures, and various analytics associated with procedures. The collaborative medical platform may furthermore intelligently generate recommendations or other medical intelligence data associated with planning or conducting a procedure, managing risk associated with a case, identifying best practices, facilitating training of practitioners, conducting clinical trials, or other purposes. Furthermore, the collaborative medical platform may enable creation and sharing of presentations, educational material, or other medical resources in a secure platform that adheres to data privacy and regulatory restrictions.

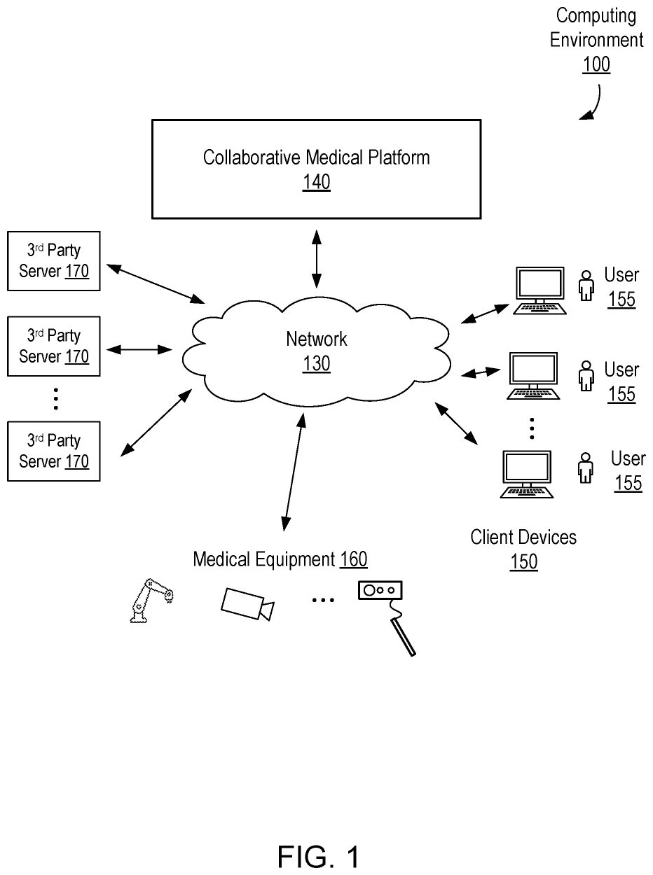

FIG. 1 illustrates an example embodiment of a computing environment 100 for a collaborative medical platform 140. The collaborative medical platform 140 may include one or more servers that are coupled by a network 130 to client devices 150 associated with users 155 of the collaborative medical platform 140, medical equipment 160, and various third-party servers 170. The collaborative medical platform 140 facilitates collaborative exchange of data between medical practitioners, patients, administrators, or other users 155 via the client devices 150 in support of preprocedural, intraprocedural, and postprocedural stages of medical cases. The collaborative medical platform 140 may furthermore facilitate access to telemetry data from medical equipment 160 (including, for example, real-time video, images, biometric sensing data, equipment control and/or status signals, etc.) that may be utilized in conjunction with performing medical procedures and managing patient cases. Furthermore, the collaborative medical platform 140 may facilitate access to various third-party servers 170 that provide external services such as, for example, electronic healthcare records (EHR) services, medical telepresence services, operating room scheduling, data analytics services, etc.

In support of a preprocedural stage of a medical case, the collaborative medical platform 140 provides an online platform that enables medical practitioners to prepare for performance of the medical procedure. Here, the collaborative medical platform 140 may provide access to various text and multimedia resources relating to the planned procedure such as video libraries, instructional information, statistical information, relevant research information, or other resources. The collaborative medical platform 140 may furthermore enable a case owner to selectively share the case with one or more remote medical practitioners to facilitate collaboration. Here, the collaborative medical platform 140 provides interfaces for facilitating interactions between remote practitioners through a case-specific content feed and enabling sharing of case information, commentary, multimedia, or other information. In an embodiment, the collaborative medical platform may automatically redact patient identifiers or selective patient information to maintain data privacy. The collaborative medical platform 140 tracks all information in association with a case identifier to maintain a complete record of case data, multimedia, collaborative content feeds, or other information collected by the collaborative medical platform 140.

In support of an intraprocedural stage of a medical case, the collaborative medical platform 140 may facilitate presentation of various information to support the procedure such as preprocedural images, models, patient data, equipment information, or other data. The collaborative medical platform 140 may furthermore facilitate a telepresence session that enables one or more remote contributors to access video, images, 3D models, equipment telemetry data, or other data streams capturing during an ongoing medical procedure. The collaborative medical platform 140 may furthermore enable remote practitioners to provide annotations or other commentary related to real-time video, images, or three-dimensional models associated with a procedure. The collaborative medical platform 140 tracks and stores all data from the procedure (including video, medical equipment telemetry, and collaborative commentary) in association with the case identifier to enable subsequent access.

In support of a post-operative stage of a medical procedure, the collaborative medical platform 140 enables medical practitioners connected with a case to collaboratively monitor data associated with a patient's recovery. For example, the collaborative medical platform 140 may provide interfaces for viewing health records associated with the patient's recovery and facilitate collaborative exchange between medical practitioners through a case-specific content feed. The collaborative medical platform 140 may furthermore perform various analytics relating to performed medical procedures based on aggregations of data. The analytics may be useful to support patient recovery, to improve future procedures, and to track the performance of medical practitioners.

The collaborative medical platform 140 may intelligently utilize data collected during preprocedural, intraprocedural, and/or postprocedural stages of a case during a different stage of the same case or other cases. For example, annotations of images or 3D models, practitioner comments from a content feed, or other information obtained during a preprocedural stage may be made available in the intraprocedural stage to aid the performing practitioner through the procedure. Analytical data relating to postprocedural data may be utilized to generate recommendations for future procedures in order to improve efficiencies and/or outcomes.

The collaborative medical platform 140 may also facilitate functions such as managing clinical trials, facilitating education training and performance tracking, facilitating broadcasts of medical-related presentations, and facilitating procedure scheduling. Beneficially, the collaborative medical platform 140 stores complete records of medical cases (including video and telemetry from procedures) in a centralized and standardized platform that naturally allows for collaboration in an online environment, where practitioners may interact from disparate remote locations. The collaborative medical platform 140 may maintain data in a manner that adheres to data privacy and compliance obligations of medical practitioners and organizations.

The collaborative medical platform 140 may furthermore employ various machine learning techniques to infer recommendations, insights, or other artificially generated contributions based on the data collected into the collaborative medical platform 140. For example, the collaborative medical platform 140 may generate a recommendation for a medical practitioner to review a case record associated with one or more historical cases captured in the collaborative medical platform 140 that relates to a similar procedure performed on a similarly situated patient. If granted appropriate permissions, the practitioner may then review the entire case record through the collaborative medical platform 140 including preprocedural information, videos or other data from the procedure itself, and postprocedural outcome data. In another example, the collaborative medical platform 140 may intelligently generate a recommendation to invite a particular medical practitioner to collaborate on a case based on that practitioner having relevant expertise, experience, and/or availability. An invitation may then be generated to the medical practitioner to enable access and collaboration on the case during at least one of the preprocedural, intraprocedural, and postprocedural stages. Furthermore, the collaborative medical platform 140 may intelligently identify and present patient risk factors relevant to procedure performance, planning, and postprocedural care. The collaborative medical platform 140 may also intelligently recommend training for medical practitioners based on their individual tracked performance and various comparative analytics.

The collaborative medical platform 140 may be implemented using on-site computing or storage systems, cloud computing or storage systems, or a combination thereof and may be implemented utilizing local or cloud-based servers, which may include physical or virtual machines, or a combination thereof. Cloud-based servers may include private cloud systems, public cloud systems, hybrid public/private cloud systems, or a combination thereof. Accordingly, the collaborative medical platform 140 may be local, remote, and/or distributed relative to the medical environments where procedures are performed and relative to the client devices 150 providing user access. Furthermore, different portions of the collaborative medical platform 140 may execute on different remote servers and various system elements of the collaborative medical platform 140 may be communicatively coupled over a network 130.

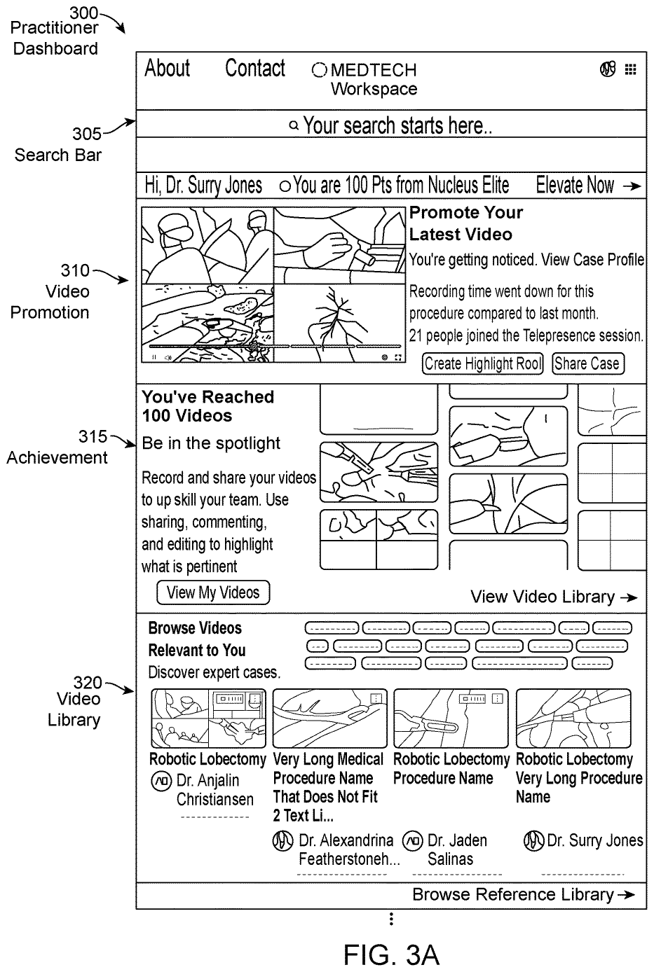

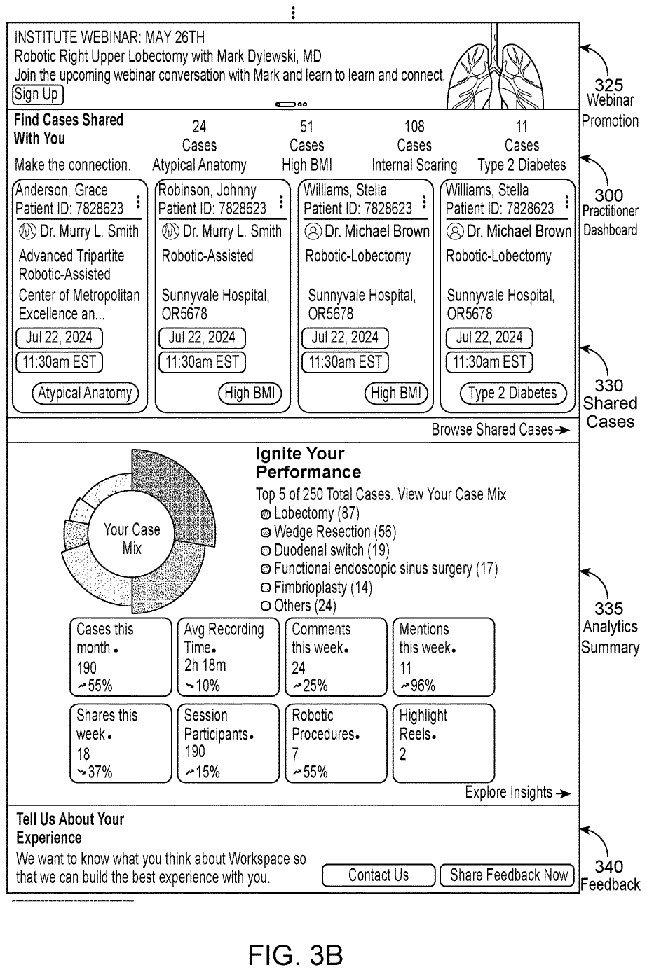

The client devices 150 may include, for example, a mobile phone, a tablet, a laptop or desktop computer, other computing device, or application executing thereon for accessing the collaborative medical platform 140 via the network 130. The client devices 150 may enable access to various user interfaces (which may comprise web-based interfaces accessed via a browser or application interfaces accessed via an application) for viewing and/or editing information associated with the collaborative medical platform 140. The client devices 150 may include conventional computer hardware such as a display, input device (e.g., touch screen), memory, a processor, and a non-transitory computer-readable storage medium that stores instructions for execution by the processor in order to carry out functions described herein. Examples of user interfaces are described in further detail below with respect to FIGS. 3-11.

The third-party servers 170 may facilitate diverse services utilized by the collaborative medical platform 140. For example, the third-party servers 170 may include various EHR systems for managing patient records, robotic control platforms for controlling surgical robots or other medical equipment, telepresence servers for facilitating telepresence services, patient scheduling systems, hospital information systems (HIS), or other servers. The third-party servers 170 may be implemented using various on-site computing or storage systems, cloud computing or storage systems such as private cloud systems, public cloud systems, hybrid public/private cloud systems, or a combination thereof.

The medical equipment 160 may include various sensors such as cameras or other imaging equipment, biometric monitors, or other sensing devices that collect data associated with a medical procedure being performed. Sensor data may include physiological or biological signals (such as pulse rate, blood pressure, body temperature, etc.), video, electrical signals representative of a state of a medical instruction, or other information. Cameras or image sensors may include still image cameras, video cameras, 3-dimensional (3D) imaging devices, or a combination thereof. The cameras can include stationary cameras in a medical environment (e.g., operating room) or may include cameras integrated into medical instruments such as endoscopic cameras. Imaging systems may include computed tomography (CT) imaging systems, medical resonance imaging (MRI) systems, X-ray systems, or other imaging equipment. The medical equipment may furthermore include a robotic device that facilitates robotically-assisted medical procedures. The robotic device may include, for example, a robotic arm or other computer-controlled mechanical device that performs or assists with a medical procedure. The robotic device may be pre-programmed to perform a certain set of steps or tasks, and/or may be manually controlled by an operator. Telemetry data associated with a robotic device may include force data, positional data, or other sensor data, control signals, fault conditions, or other data relating to operation of the robotic device during a procedure. The medical equipment data may be streamed to the collaborative medical platform 140 in real-time or may be stored on a third-party server 170 and later uploaded to the collaborative medical platform 140.

The network 130 comprises communication pathways for communication between the collaborative medical platform 140, the medical equipment 160, the client devices 150, and the third-party servers 170. The network 130 may include one or more local area networks and/or one or more wide area networks (including the Internet). The network 130 may also include one or more direct wired or wireless connections (e.g., Ethernet, WiFi, cellular protocols, WiFi direct, Bluetooth, Universal Serial Bus (USB), or other communication link).

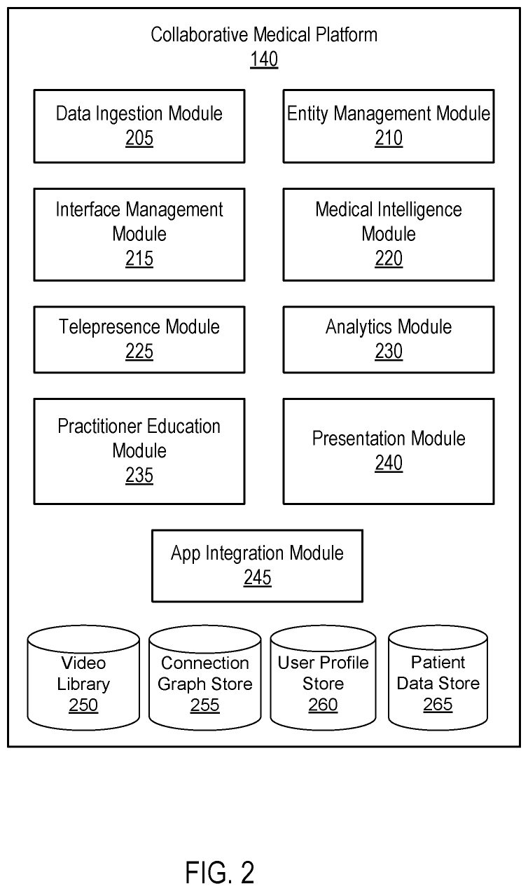

FIG. 2 is a block diagram showing an example architecture of an embodiment of the collaborative medical platform 140. In the embodiment of FIG. 2, the collaborative medical platform 140 includes a data ingestion module 205, an entity management module 210, an interface management module 215, a medical intelligence module 220, a telepresence module 225, an analytics module 230, a practitioner education module 235, a presentation module 240, an application integration module 245, a video library 250, a connection graph store 255, a user profile store 260, and a patient data store 265. In other embodiments, the collaborative medical platform 140 includes different or additional functional blocks than those shown in FIG. 2. Further, in some embodiments, a single functional block provides the functionality of multiple functional blocks shown in FIG. 2.

While in one embodiment, the illustrated functional blocks may execute entirely within the collaborative medical platform 140, alternative embodiments may include various modules or discrete functions of modules being executed by one or more third-party servers 170. Here, the collaborative medical platform 140 may interact with a third-party server 170 via an application programming interface (API) to enable the collaborative medical platform 140 to request and utilize services provided by the third-party servers 170 to facilitate any of the functions described herein. For example, in an embodiment, electronic health records may be provided by a third-party server 170. Here, the collaborative medical platform 140 may query the third-party server 170 for relevant data but does not necessarily locally store complete patient records. Furthermore, third-party servers 170 may facilitate services such as telepresence sessions, presentation creation, access to video resources, three-dimensional model generation, or other aspects of the functions of the collaborative medical platform 140 described herein.

The data ingestion module 205 ingests various medical data used by the collaborative medical platform 140. The data ingestion module 205 may be electronically coupled to one or more external servers, databases, or other data sources that supply the medical data. The medical data may include, for example, profile data for patients (e.g., demographic information, health history, etc.), medical professionals (e.g., expertise, experience, etc.), or facilities, information about medical conditions, procedures, and medications, information about robotic systems, imaging systems, intervention tools, or other medical equipment, information about post-procedural outcomes, or other medical information discussed herein.

The data ingestion module 205 may aggregate data from various input data sources. For example, the data ingestion module 205 may obtain medical data from conventional electronic health records (EHR) systems. Here, the data ingestion module 205 may perform various pre-processing to normalize data to a standardized format used by the collaborative medical platform 140. For example, medical records may be organized in a database structure that includes values (strings, numerical values, binary values, or other data types) assigned to each of a set of predefined information fields.

The data ingestion module 205 may furthermore interface with one or more imaging systems to ingest preprocedural, intraprocedural, or postprocedural images, video, or three-dimensional models associated with patients. For example, the data ingestion module 205 may obtain and store X-ray images, magnetic resonance imaging (MRI) images, computed tomography (CT) scan images, visible light images, near infrared fluorescent (NIRF) images, or other medical images, video, or three-dimensional models derived from them. Image data may furthermore include image or video data from one or more cameras present in a medical environment where a medical procedure is being performed, such as one or more overhead cameras and/or one or more endoscopic cameras. Imaging data may include associated metadata such as telemetry data from one or more medical instruments used to perform the medical procedure, annotations or commentary associated with the video received from one or more medical practitioners associated with the medical procedure, segmentation data associated with dividing a video into segments relating to different steps of a procedure, or other information relating to image or video data.

To simplify subsequent retrieval and review of video of a medical procedure along with associated metadata, the data ingestion module 205 may perform various preprocessing and indexing of the content and associated metadata. For example, the data ingestion module 205 indexes video of a medical procedure with associated metadata to correlate different metadata with different portions of the video, synchronize videos associated with the same medical procedure, or perform various encoding or reformatting of video data. Videos may furthermore be automatically segmented and indexed into video segments corresponding to different steps of a procedure.

The data ingestion module 205 may furthermore integrate with various robotic platforms or other medical equipment to obtain telemetry data associated with procedures. For example, the data ingestion module 205 may obtain various sensor data from sensors utilized during medical procedures, identifying information associated with medical equipment, control data associated with control a robotic platform or other medical equipment, or other data generated from medical equipment in associated with performed medical procedures.

The data ingestion module 205 may furthermore provide interfaces accessible via the client device 150 for ingesting data input directly into the collaborative medical platform 140. For example, the data ingestion module 205 may present various forms or freeform entry elements to enable entry of medical information relevant to operation.

In an embodiment, the data ingestion module 205 may manage data in a manner consistent with various compliance and privacy policies. For example, the data ingestion module 205 may enable removal or redaction of portions of received data to preserve privacy of a patient when the data is used for purposes in which patient identification is not necessary.

The entity management module 210 manages presentation of entity pages associated with different entities affiliated with the collaborative medical platform 140 and manages connections between entities. Entities may include, for example, users 155 (which may medical practitioners, patients, administrators, etc.), medical cases associated with procedures, facilities, medical equipment 160, files or media content, events (e.g., conferences), presentations, training modules, or other data objects. Entity pages may comprise web pages accessible via a web browser of the client device 150 or may comprise pages of a desktop or mobile application installed on a client device 150.

Each entity page for an entity may enable viewing of information associated with the entity and/or interactions with the entity. For example, each user 155 of the collaborative medical platform may have a dedicated page that provides information about the user 155 such as identifying information, role (e.g., surgeon, nurse, executive, administrator, patient, etc.) profile information (e.g., biography, credentials, etc.), assigned cases, procedure histories, connections to other users or cases, scheduling information, or other user-specific data. An entity page for a patient (whether or not the patient is a user 155 of the collaborative medical platform 140) may include patient profile information, health history, planned procedures, risk factors, or the medical information associated with the patient. An entity page for a medical case may include information about a patient associated with the case, descriptive information about a medical procedure (such as a type of medical procedure) associated with the case, a medical environment where the medical procedure is to be performed, other descriptive information about the medical procedure, a status of the procedure (e.g., preprocedural stage, intraprocedural stage, or postprocedural stage), or other information relevant to a medical case. Pages may furthermore include various interactive elements (e.g., content feeds) that enable users to share and interact with data associated with that entity as will be further described below.

The entity management module 210 also organizes pages and associated data received into the collaborative medical platform 140 into a connection graph (stored to the connection graph store 255) that captures relationships between different entities and associated data. Some connections may be configured as default connections, while other connections may be created based on specific actions from users 155. For example, users 155 may be connected by default to other users 155 (with at least viewing permissions) within the same organization. Alternatively, connections may be generated only when a user 155 expressly invites another user 155 to connect and the receiving user 155 accepts the connection request. Connections between medical practitioners and medical cases may similarly be created by default or in response to invitations to create a connection. For example, a default connection may be created between an entry for a planned medical procedure and a medical practitioner assigned responsibility for the procedure. Alternatively, all medical practitioners within an organization or within a relevant department may become connected to a planned procedure as a default. In other scenarios, a user may share a medical case with one or more other medical practitioners to generate a connection request that invites the other medical practitioners to collaborate with on the medical case. Accepting the connection request may then create a connection between the invited practitioner and the medical case. Supplemental connections may also automatically be generated (e.g., between the owner of the procedure and the invited contributor). Connections may furthermore be created between users 155 and individual videos, files, presentations, or other data objects. For example, a user 155 that creates or owns a video may share the video with one or more other users 155 to grant access rights to the video.

Connections between entities may be of diverse types and may be governed by different permissions. Generally, pages may be accessed only by users having appropriate access permissions. Different permission levels may dictate distinct levels of access for different pages. For example, depending on user-specific permissions for a particular page, the user may be permitted or blocked from accessing the data, editing the data, commenting or annotating the data, deleting the data, or performing other modifications. In an embodiment, a page may have a page owner with the highest level of access permissions. Generally, a medical practitioner may be the page owner for their own profile page and for procedures for which they have primary responsibility. Pages associated with facilities, medical equipment, or other entities may variably be owned by an assigned medical practitioner. Non-owners may have distinct levels of access to pages depending on the configured permissions. Permissions may be granted by the page owner or by another user that has appropriate permissions to assign or relinquish permissions to other users.

Based on different connections available to different users 155, the collaborative medical platform 140 enables a personalized experience for each user 155. For example, upon logging into the collaborative medical platform 140, a user 155 may be presented with personalized interfaces that relate to their connections to other users 155, medical cases, videos, presentations, or other content hosted by the collaborative medical platform 140.

An interface management module 215 manages content associated with various interfaces hosted by the collaborative medical platform 140 and accessible via the client devices 150. As described above, the interface management module 215 may manage pages associated with the various entities managed by the collaborative medical platform 140 including users 155 (which may include medical practitioners, patients, administrators, etc.), medical cases associated with procedures, facilities, medical equipment 160, files or media content, events (e.g., conferences), presentations, training modules, or other data objects. Access to different pages by a specific user 155 may be dependent on that user's connections and permissions configured in the connection graph store 255. Furthermore, patient data may be pseudonymized for viewing by certain other users (dependent on the type of connection and/or permission) such that the patient data cannot be attributed to a specific individual.

A medical case page associated with a medical case may include information organized into preprocedural, intraprocedural, and postprocedural stages. At the preprocedural stage, a medical case page may include information about the patient, the procedure being performed, and the medical practitioner performing the procedure. The interface management module 215 may furthermore provide access to various analytical information (e.g., generated by the analytics module 230 described below) such as risk factors for the patient, experience/expertise of the medical practitioner, outcomes for the type of procedure being planned, or other data. At the intraprocedural stage, the medical case page may provide access to a telepresence session to enable remote collaborators to remotely collaborate with respect to an ongoing procedure. At a postprocedural stage, the medical case page may include information about the patient treatment plan, risk factors, follow up visits, or other postprocedural information.

Some entity pages in the collaborative medical platform 140 may include content feeds to facilitate collaboration between users 155. Content feeds may include various content (e.g., posts) such as text-based commentary, images, video, three-dimensional models, or other multimedia content relating to a medical case. Content may be directly posted to a page associated with a medical case or a post may comprise links to content stored by the collaborative medical platform 140 or on an external server. Posts may be grouped into conversations that hierarchically track the relationships between posts. For example, posts may be made as original posts (which start a new conversation) or as replies to existing posts (which become part of the conversation).

In an example use, a user 155 may invite one or more other users 155 to collaborate on a medical case and thereby gain access to a case page for the medical case. A content feed on the page enables the collaborating users 155 to post to the case page in association with the medical case. The content feed may therefore enable discussion about the procedure to be performed discussion of risk, best practices, or other information that may be useful to the practitioner performing the procedure. Furthermore, the contributing users 155 may post videos or three-dimensional models (or links to content) relating to historical procedures for similarly situated patients. Additionally, contributing users 155 could share links to entity pages associated with past procedures that may be of relevance, to enable a performing medical practitioner to view historical content feeds associated with those procedures. Patient data may optionally be pseudonymized when shared with other users (dependent on the type of connection and/or permission) such that the patient data cannot be attributed to a specific individual.

Content feeds may furthermore be utilized in relation to an ongoing procedure during a real-time telepresence session as discussed in further detail below. Here, a content feed may be presented as a real-time chat window that enables contributors to comment during a procedure, share video, images, or other media, provide links to relevant resources, or otherwise contribute content during the course of procedure.

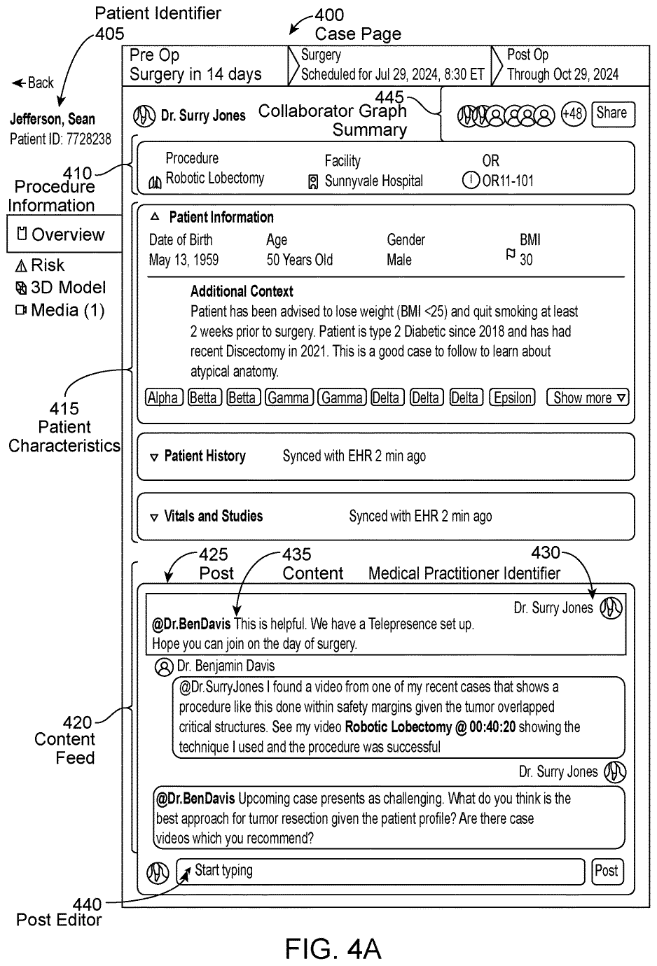

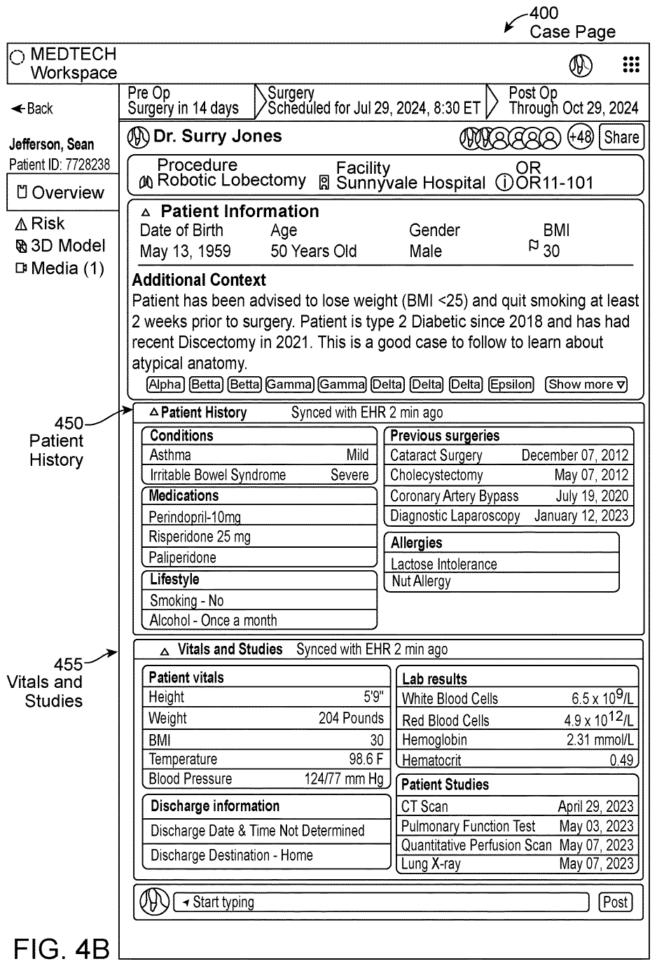

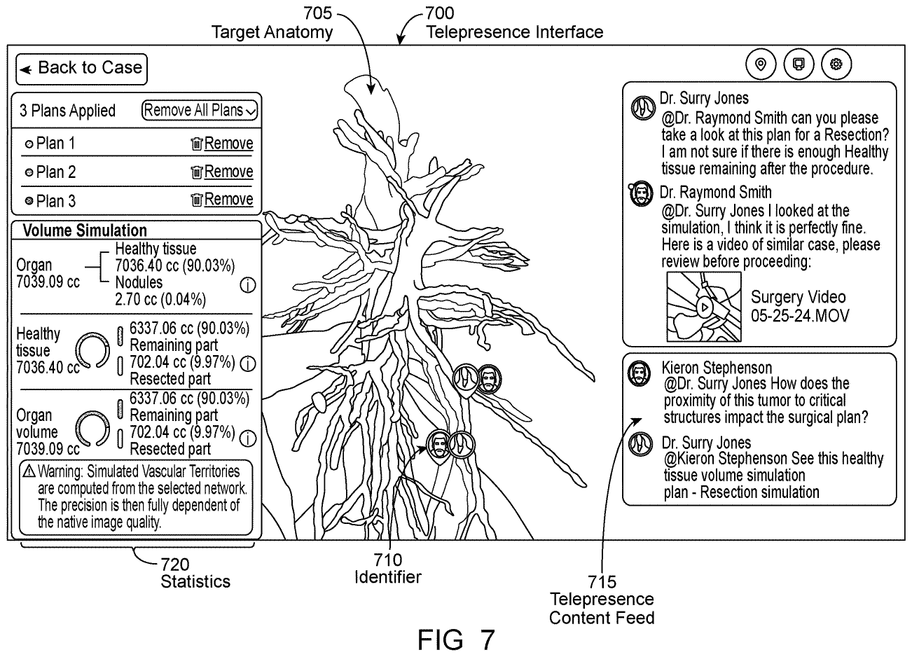

In a postprocedural stage, a content feed may be utilized by contributors to discuss postprocedural treatments, patient recovery, risk management, or other information relevant to patient recovery. Examples of content feeds are provided in FIGS. 4A and 7 which are described in further detail below.

The medical intelligence module 220 generates medical intelligence data that may be automatically added to content feeds or otherwise made available in the context of the collaborative medical platform 140. For example, the medical intelligence module 220 may automatically contribute posts to a content feed for a medical case that an artificial intelligence agent infers is relevant. Artificially generated posts may mimic posts provided by human contributors and may include text-based commentary, multimedia, links, etc. Medical intelligence data may be generated during a preprocedural stage, during a procedure, or during a postprocedural stage.

In an example implementation, the medical intelligence module 220 may include one or more machine-learned models trained to generate content that the models infer to be relevant to a particular medical case or more generally relevant to a user 155. In one implementation, the machine learned model generates an embedding for a medical case based on descriptive information about a medical procedure, characteristics of the patient on whom the medical procedure is to be performed, characteristics of medical practitioner performing the procedure, posts in the content feed, or other information available in the collaborative medical platform 140. The medical intelligence module 220 determines measures of similarity (e.g., cosine similarity, dot product) between the embedding for the medical case and embeddings for other content available in the collaborative medical platform 140 and that can be included in automated posts. The medical intelligence module 220 may then generate posts and/or select content for posts based on similarities of the embeddings. The medical intelligence module 220 may furthermore employ various Large Language Models (LLMs) to analyze text-based content associated with a medical case and artificially generate relevant natural language content for the content feed. Machine learning models may furthermore include one or more neural networks (such as convolutional neural network (CNN), artificial neural network (ANN), residual neural network (ResNet), or recurrent neural network (RNN)), regression-based models, generative models, or other type of machine-learned model capable of achieving the functions described herein.

In an example use case, the medical intelligence module 220 may identify one or more historical medical cases that are similar to a current medical case and automatically generate a link to a case page for the related case. A medical practitioner may then view videos, models, or other recorded data associated with the related medical case to help the practitioner prepare for a procedure. In other examples, the medical intelligence module 220 may automatically respond to a question posed by a user in the content feed. For example, the medical intelligence module 220 may operate like a chatbot that intelligently responds to text-based queries. In further embodiment, the medical intelligence module 220 may generate a recommendation to invite a specific medical practitioner to collaborate on a medical case based on relevant expertise and experience. A user may then select to invite the recommended collaborator to collaborate on the medical case based on the artificially generated recommendation.

The telepresence module 225 facilitates a telepresence session during a procedure. The telepresence session may be joined by one or more collaborators that have been invited to collaborate on the medical case and enable the other users 155 to remotely access video, telemetry data from one or more medical instruments, or other real-time data captured during a medical procedure. As described above, a content feed may also be displayed in association with the telepresence session to enable contributors to comment or share multimedia or links relevant to the procedure.

The telepresence module 225 may furthermore enable contributors to provide real-time annotations on images, video, three-dimensional models, or other visual content of anatomy relevant to an ongoing procedure. For example, a contributor may mark locations in the visual content in association with provided comments. The telepresence module 225 may furthermore enable contributors to add overlaid drawings, highlighting, or other visual indicators during an ongoing telepresence session.

In an embodiment, the telepresence module 225 may enable remote contributors to take control of medical equipment 160. For example, a remote contributor may access a control interface that provides control elements for controlling a position or orientation of a camera, controlling a robotic arm, setting a configuration of a sensing device, or performing other control functions of medical equipment.

Upon completing a telepresence module 225 the recorded video, telemetry data, content feed, annotations, and other captured data may be stored in association with the procedure. This information may be later accessed by users 155 of the collaborative medical platform 140 (with appropriate permissions) and/or may be utilized by the medical intelligence module 220 to further train machine learning models and/or generate inferences.

The analytics module 230 facilitates generation of various statistics, metrics, or other analytics associated with information stored in the collaborative medical platform 140. Analytics may generally be created based on a set of filtering parameters that yield some subset of data records for aggregating, and a combining function that specifies how the filtered data should be combined. The filtering parameters may filter medical procedure data based on data fields such as patient data, medical practitioner data, facility, procedure type, medical equipment used, etc. The combining function may comprise, for example, an averaging function, a median function, a histogram function, or other function. A specific analytics function may result in a single output value or a series of values over one or more dimensions. Series outputs may be visually represented in a table, chart, graph, or other visual output.

For example, the analytics module 230 may generate metrics describing an average length of time for a specific medical practitioner or a group of medical practitioners to complete a medical procedure. Average times for various procedures performed by the same medical practitioner or group of practitioners may be presented together with similar metrics for other medical practitioners for comparison purposes. In another example, the analytics module 230 may generate metrics describing a number of times a medical practitioner has historically performed a specific type of medical procedure. Such counts could be further aggregated to indicate percentages that reflect how many times a medical practitioner has performed each different type of medical procedure out of a total number of procedures performed.

In further embodiments, the analytics module 230 may generate analytics based on interactions of the medical practitioner in the collaborative medical platform 140. For example, statistics can be derived based on counts of posts, comments, or other content contributed by a medical practitioner to the collaborative medical platform 140. Such analytics may be expressed in terms of counts of interactions, frequency of interactions, or other aggregations. These analytics could furthermore be separately aggregated based on whether interactions relate to preprocedural, intra-procedural, or post-procedural phases of procedures.

In an embodiment, the analytics module 230 may generate analytics based on specific filtering and/or combining functions specified by a user 155 of the collaborative medical platform 140. Additionally, the analytics module 230 may include various preset analytics that may be generated without necessarily receiving specific user inputs. Furthermore, in some embodiments, the medical intelligence module 220 may automatically generate analytics that it infers will be relevant to a specific user 155.

In some embodiments, the analytics module 230 may generate analytics based on any aspects of the collective case data including preprocedural data, telepresence sessions data (including recorded video, telemetry data, in-session content feed data, etc.), and postprocedural data. Analysis associated with telepresence session data may include performing various video processing, content recognition, or other advanced image processing techniques to extract useful information from videos. Furthermore, the analytics module 230 may leverage various medical intelligence data generated from the medical intelligence module 220 to generate analytics.

The practitioner education module 235 manages and stores training data for medical practitioners associated with medical procedures. Examples of training data managed by the practitioner education module 235 may include training videos relating to performing medical procedures, articles about medical procedures, articles about patient diagnosis, articles about courses of treatment for a patient recovering from a medical procedure, best practices for a medical procedure, training manuals for medical procedures, instructional material for one or more medical instruments used in a medical procedure, digital training modules, webinars, or other information for training medical practitioners in relation to medical procedures.

In an embodiment, the practitioner education module 235 may automatically recommend training modules or other training materials to medical practitioners based on monitored performance of a medical practitioner, which may be compared against various averages, metrics, other standards. For example, the practitioner education module 235 may detect that a particular medical practitioner generally performs a specific type of procedure in a timeframe that is significantly higher or lower than the global average. This may suggest that the practitioner is employing non-standard procedures and the practitioner education module 235. The practitioner education module 235 may detect these discrepancies and recommend training related to the procedure. In some embodiments, one or more machine learning models applied by the medical intelligence module 220 may infer training for the practitioner education module 235 to recommend to a practitioner. For example, a machine learning model may be trained to infer, based on characteristics and performance history of a medical practitioner, that completing a specific training course is predicted to enable the practitioner to improve performance in a manner that increases efficiency and/or improved patient outcomes.

The presentation module 240 leverages stored information associated with a completed medical procedure to facilitate generation of presentations for education, research, training, or other purposes. Presentations may be in the form of slide decks, posters, videos, animations, or other multimedia content. Presentations may incorporate various multimedia (e.g., video, images, three-dimensional models, and associated metadata), patient record data, medical equipment telemetry data, information from content feeds, analytics, or other information generated and/or stored by the collaborative medical platform 140.

In an embodiment, the presentation module 240 may maintain one or more presentation templates for generating presentations. The template may include pre-formatted content with various information fields that may be automatically populated from a set of records. For example, a practitioner wanting to prepare a presentation relating to a set of recently performed procedures may specify the set of procedures to include in the presentation, and the presentation module 240 may automatically populate the presentation based on the data stored in association with those procedures. pages, with each page associated with one or more types of data about the completed medical procedure. In some embodiments, the presentation module 240 may apply one or more trained machine learned models to automatically generate and/or recommend presentation content that may be of interest to a medical practitioner. In further embodiments, the presentation module 240 may intelligently automatically de-identify patient data included in the presentations.

The presentation module 240 may furthermore include various editing tools for creating, viewing, and editing presentations. For example, the editing tools may enable editing of text, video, images, animations, three-dimensional models, or other content for including in a presentation.

In an embodiment, presentations may be presented through a presentation module 240 directly without data associated with the presentation being exported externally to the collaborative medical platform 140. For example, the presentation module 240 may enable live streamlining of a presentation during a telepresence session to a set of invited attendees. The invited attendees may be limited to users 155 of the collaborative medical platform 140 or may include outside attendees that may gain access via an external link. Sharing presentations in this manner enables practitioners to maintain data privacy and compliance and avoid issues that may arise when externally exporting medical data.

The application integration module 245 manages integration of applications with the collaborative medical platform 140. Applications may be utilized to add additional optional functionality to the collaborative medical platform 140. For example, applications may enable integration with a specific EHR system, scheduling system, or other existing medical system. Applications may furthermore enable users to selectively add specific functionality beyond the core features of the collaborative medical platform 140. The application integration module 245 may allow third parties to create applications that interface with the collaborative medical platform 140 and make these applications available to add.

The application integration module 245 may maintain a catalog of applications capable of interfacing with the collaborative medical platform 140 and may provide interfaces to enable users 155 to selectively add applications for integration. In various embodiments, applications identified by the application integration module 245 have been authorized or approved for installation by an administrator of the collaborative medical platform 140, allowing regulation of the applications capable of executing on the collaborative medical platform 140.

Additionally, the application integration module 245 may include one or more application programming interfaces (API) for an application installed through the application integration module 245. An API for an application provides functionality for exchanging data between the application and one or more components of the collaborative medical platform 140, simplifying data exchange between the application and other portions of the collaborative medical platform 140.

The video library 250 stores videos of various medical procedures, training presentations, simulations, or other medical videos and metadata associated with the video. Examples of metadata associated with video of a medical procedure may include telemetry data of one or more medical instruments received in conjunction with the video, comments or annotations received from one or more medical practitioners through a surgical interface during the medical procedure included in the video, segmentation data that divides the video into temporal segments relating to different step of a procedure, profile information (e.g., age, body mass index, gender, etc.), associated with the patient in the video, or other information supplementing the video.

The video library 250 may store videos in an indexed database that indexes videos based on various metadata. The video library 250 can then be browsed or searched via a video library interface to identify videos of relevance. The metadata associated with videos may include permissions stored in the connection graph store 255 that controls which users 155 have access to different videos. For example, a video in the video library 250 may be accessible only to users 155 that the video has been expressly shared with or that otherwise has viewing permissions for the video.

The connection graph store 255 comprises a database that stores information describing connections between entities or other objects (e.g., videos or other multimedia) managed by the collaborative medical platform 140. For example, as described above, the connection graph store 255 stores connections between users 155, connections between users 155 and procedures, connections between users 155 and multimedia content or other objects, or other connections between data entities of the collaborative medical platform 140.

The user profile store 260 stores profile data for users 155 of the collaborative medical platform 140. A user profile for a medical practitioner includes descriptive information such as a name of the medical practitioner, contact information for the medical practitioner, credentials or certifications of the medical practitioner, biographical information for the medical practitioner, types of medical procedures capable of being performed by the medical practitioner, medical facilities affiliated with the medical practitioner, operating room preferences (such as patient positioning, equipment setup, preferred instrumentation, typical procedure step order, etc.), equipment configuration preferences (e.g., ergonomic settings for a robot console), or other information describing the medical practitioner. Aspects of the user profile could be inferred using machine learning techniques. For example, a practitioner's preferred instrumentation or step order may be inferred from application of a machine learning model trained to infer such preferences based on observed historical data. Additionally, a user profile for a medical practitioner includes medical procedures performed by or to be performed by the medical practitioner, as well as information describing the medical procedures. For example, the user profile identifies different types of medical procedures to be performed by, or performed by, the medical practitioner, and may include characteristics for each medical procedure (e.g., a length of time to complete the medical procedure, a number of times the medical practitioner performed a type of medical procedure matching the medical procedure, etc.). Further, one or more of the metrics determined by the analytics module 230 for the medical practitioner, as further described above, may be included in the user profile for the medical practitioner.

The patient data store 265 includes a patient profile for each patient associated with medical cases. A patient profile includes characteristics of a corresponding patient, which may be obtained from an electronic health record for the patient or may be provided via input from a medical practitioner. Characteristics of a patient include demographic information about the patient, medical conditions of the patient, medical procedures previously performed by the patient, allergies of the patient, contact information for the patient, current or prior prescriptions for the patient or other medically relevant information about the patient. A patient identifier is associated with a patient profile to uniquely identify the patient profile.

All of the data stored to the collaborative medical platform 140 (or otherwise made available through the collaborative medical platform 140) may be stored, presented, and in some cases restricted in a manner that ensures compliance with various data privacy and protection regulations.

FIGS. 3A-3B illustrate an example practitioner dashboard 300. FIG. 3A shows an upper portion of the dashboard 300 while FIG. 3B shows a lower portion of the dashboard 300 (which may be continuously scrollable). The practitioner dashboard 300 may operate as a home landing page for a medical practitioner upon logging into the collaborative medical platform 140. The practitioner dashboard 300 may include various content sections, at least some of which may be specifically targeted to the practitioner. A search bar 305 enables input of text-based search queries for searching content available in the collaborative medical platform 140 (e.g., case pages, other user pages, videos, presentations, etc.). In response to inputting a search query, a list of results may be displayed with links to content matching the search query. A video promotion section 310 shows a video recently added by the practitioner with user interface tools to enable the practitioner to promote the video by sharing it with other users, create a highlight reel, or view various statistical information about the video. An achievement section 315 presents an achievement relating to use of the collaborative medical platform 140. In this example, the achievement section 315 highlights that the user has recently reached 100 videos and provides links to view the user's videos and access a video library. Other examples of achievements in the achievement section 315 could relate to number of cases managed, time using the platform 140, number of connections, count of frequency of interactions, or other usage achievements. The video library 320 includes video thumbnails, video tags, or other links to enable browsing of videos selected as potentially relevant to the medical practitioner. For example, relevant videos may be selected that relate to past or upcoming procedures associated with the medical practitioner, based on a history of videos viewed by the medical practitioner, based on a practice area or other profile information for the medical practitioner, or other factors. The webinar promotion section 325 includes a promotional banner for an upcoming webinar that will be viewable within the collaborative medical platform 140. The webinar may be identified as being of potential interest to the medical practitioner based on, for example, the subject matter of the webinar, the host of the webinar, or other factors. The shared cases section 330 provides summary information and links to case pages that have been shared with the medical practitioner. Examples of case pages are described in further detail below. The analytics summary 335 includes example analytics associated with the medical practitioner's usage of the collaborative medical platform 140, procedures performed by the medical practitioner, or other analytics data derived from information stored in the collaborative medical platform 140. The analytical data may be presented in one or more visual representations such as a graph or chart. The feedback section 340 provides links to enable the medical practitioner to send feedback to an administrator of the collaborative medical platform 140.

FIGS. 3A-3B illustrate just one example of a practitioner dashboard 300. The types of content presented in the practitioner dashboard 300 may be different for different practitioners and/or may dynamically change over time for the same medical practitioner. Some of the sections may be fixed and always appear upon accessing the dashboard 300 (e.g., the search bar 305, video library 320, shared cases 330, analytics 335, and feedback sections 340), while other sections (e.g., video promotion 310, achievement 315, webinar promotion 325) may be dynamically inserted only in certain contexts. For example, webinar promotions 325 may be presented only when an upcoming webinar deemed to be of sufficient interest is upcoming. Achievements 315 may similarly be displayed only when a relevant achievement has recently been achieved. Furthermore, the dashboard 300 could be customized by the user to display desired sections in a configured order. The various sections 305, 310, 315, 320, 325, 330, 335, 340 when present, may furthermore be presented in different order in different contexts.

FIGS. 4A-4B shows an example embodiment of a medical case page 400 associated with a specific case. In FIG. 4A, the “patient history” and “vitals and studies” sections are collapsed such that their contents are hidden from view. These sections can be expanded to view additional information as shown in FIG. 4B. The case page 400 may include various user interface elements such as a patient identifier 405, procedure information 410, patient characteristics 415, a content feed 420, a post editor 440, and a collaborator graph summary 445.

The patient identifier 405 displays information uniquely identifying a patient on which the medical procedure is intended to be performed. The patient identifier 405 may include a name of the patient and/or a unique patient identifier (e.g., a numeric code or alphanumeric code that does not identify the patient by name). The patient identifier 405 may correspond to an identifier from an EHR system and/or an identifier used in the collaborative medical platform 140 that is associated with profile data for the patient.

The procedure information 410 displays information describing the medical procedure. Example procedure information 410 may include a type of the medical procedure, a medical facility where the medical procedure is to be performed, an operating room assigned to the medical procedure, or other information. The type of medical procedure may be selected from a predefined standardized list of procedures. The type of medical procedure may furthermore indicate both the general procedure category (e.g., lobectomy) and a specific approach to the procedure (e.g., “robotic lobectomy”).

The patient characteristics 415 may include various patient information stored to a patient profile corresponding to the patient identifier 405. Examples of patient characteristics 415 that may be displayed can include demographic information (e.g., date of birth, gender, age etc.), physiological data (e.g., body mass index, weight, etc.), results of one or more medical tests performed on the patient, medicines prescribed to the patient, prior medical procedures performed on the patient, prior diagnoses for the patient, prescriptions for the patient, comments from one or more medical practitioners about the patient, or other information about the patient. The patient characteristics 415 are displayed in different sections in some embodiments, with each section including a different type of patient characteristics 415. For example, a section includes demographic information, an additional section includes physiological data, and another section includes medical test results. The case page 400 may enable distinct categories of patient information to be made visible or hidden from view for ease of viewing. The case page 400 may furthermore provide flags or other visual indicators to visually identify information that may be highly relevant to a medical practitioner performing or collaborating on the procedure. For example, in the illustrated patient information, a visual indicator appears next to the patient's BMI to indicate that the BMI is higher than recommended. Furthermore, the case page 400 could include information indicating patient consent for data processing or sharing of information, which may be case-specific or universally applicable to the patient.

The content feed 420 includes one or more posts 425 received from medical practitioners that may be connected to the case in the collaborator graph. Each post 425 may include an identifier 430 of a medical practitioner that provided the post 425 and content 435 provided by the posting medical practitioner. The content 435 may include various data types such as text data, video data, audio data, links to other content, or other types of data. The content feed 420 may display the posts 425 in chronological order or in reverse chronological order.

Posts 425 may be grouped into one or more conversations that may include nested posts. Here, a conversation may include an original post and one or more reply posts that are posted in response to the original post, or in response to another reply post within the conversation. A practitioner making a post may submit the post as a new original post (thus starting a new conversation) or may select to make a reply post to a specifically selected existing post (which may itself be an original post or a reply post).

A post editor 440 includes a text box the enables a contributor to add a post (an original post or reply) to the content feed 420. The post editor 440 may enable entry of text data, audio data, video data, image data, links, or some combination thereof. In response to receiving an input via the post editor 440, the case page 400 formats and posts the post to the content feed 420.

The case page 400 may furthermore display a collaborator graph summary 445 that summarizes the list of contributors connected to the case. In the illustrated example, the summary 445 shows profile pictures for several contributors, and a general indicator showing how many additional contributors are connected to the case. The collaborator graph summary 445 furthermore may include a share button to enable a case owner (or other contributor with sharing permissions) to share the case with additional contributors.

As illustrated in FIG. 4B, the patient history section 450 (when expanded) may show additional information about the patient such as conditions, medications, lifestyle information, and previous surgeries, allergies. The vitals and studies section 455 may show information such as patient vitals, lab results, discharge information, and patient studies information.

While FIGS. 4A-4B represents one example case page 400 associated with a case, the case page 400 may have additional interface screens (which may be organized into tabs) associated with other aspects of the case. For example, the case page 400 relates to an “overview” tab, but a case may be associated with other tabs that provide additional details about the case. For example, a case may include a risk profile tab that enumerates various identified risks associated with the case. Another tab may include one or more 4D models of the patient's anatomy relevant to the procedure, and a media tab that includes various media relevant to the procedure such as images or video (pre-operative, intra-operative, and/or post-operative).

The case page 400 may furthermore include a timeline summary that indicates whether the patient is in the pre-operative, intra-operative (e.g., surgery), or post-operative stage. The different elements of the timeline summary may furthermore operate as tab selection controls to switch between interface screens associated with the various stages of a procedure. For example, an intra-operative tab may provide information relevant to the intra-operative stage such as providing access to real-time intraoperative video, images, medical equipment telemetry data, contributor commentary, or other information. The post-operative tab may provide information relating to post-operative recovery progress, complications, post-operative medication, scheduled follow-up visits, or other information.

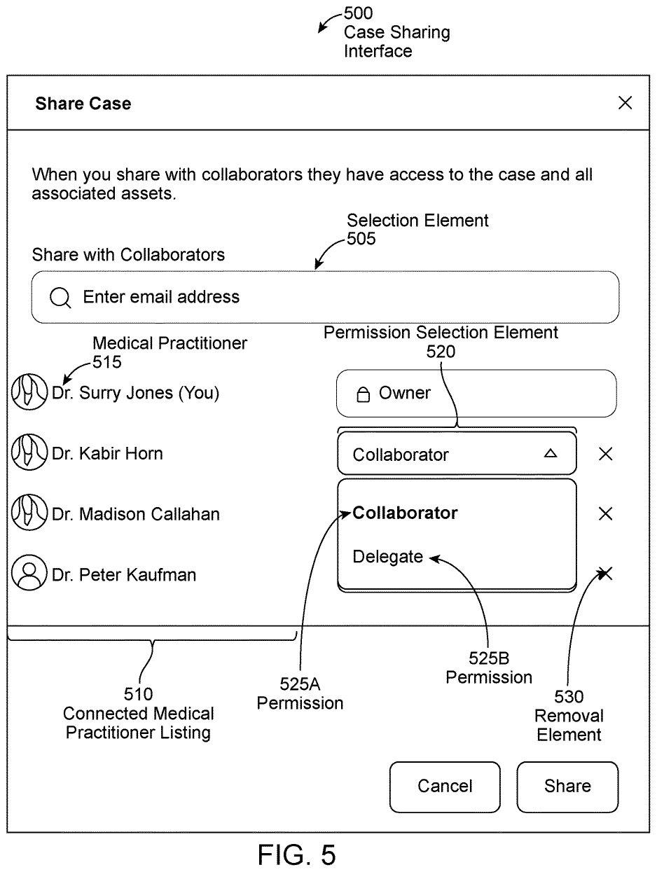

FIG. 5 shows an example embodiment of a case sharing interface 500 for sharing a case with one or more contributors. Adding a contributor to a case may generate a connection between the contributor and the case and between the contributor and the case owner. The case sharing interface 500 includes a selection element 505 for receiving identifying information to identify a desired contributor. For example, the selection element 505 may receive an email address, name, a username, or another identifier of a medical practitioner or other requested contributor. In some embodiments, upon selecting identifying information for a desired contributor, the case sharing interface 500 may display all or a portion of profile data for the requested collaborator to enable the requestor to confirm if the matched profile data is the intended collaborator. The case sharing interface 500 then enables the requestor to confirm or decline selection of a collaborator and interact with a permission selection element 520 to set a desired permission level for the requested collaborator. Here, the permission level may place limits on an invited collaborator's access to data about the case and/or may limit actions the collaborator is permitted to perform in association with the case. In an example embodiment, the permission level may be selected between a “collaborator” level 525A and a “delegate” level 525B.

In response to receiving inputs to select a requested collaborator and set a desired permission level (via the permission selection element 520), the case sharing interface 500 may send an invitation to the requested contributor (e.g., via an email, text message, phone call, portal message, or other communication mechanism) to enable the requested collaborator to accept or decline the request. If the request is accepted, the case sharing interface 500 may add the identifier or other information for the new collaborator to a connected medical practitioner listing 510 that lists the contributors added to the case. For example, the illustrated example shows a connected medical practitioner listing 510 that includes the case owner 515 and three additional contributors that have been added to the case.

The case sharing interface 500 may furthermore enable the case owner to change permission levels of existing contributors in the connected medical practitioner listing 510. Furthermore, the case sharing interface 500 may include removal elements 530 associated with each contributor in the connected medical practitioner listing 510 that enables removal of a contributor from the case. Selection of a removal element 530 may remove the stored connection in between the practitioner and the case, such that the practitioner no longer has access to the case.

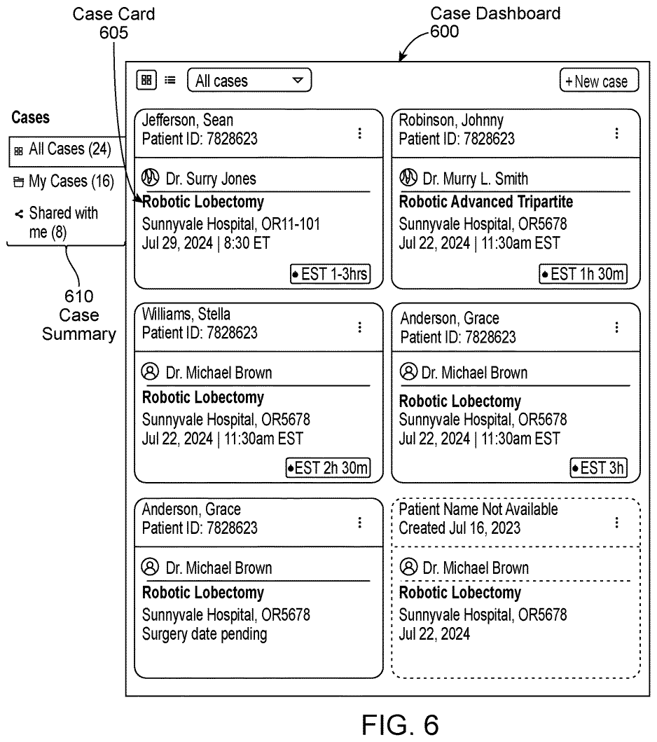

FIG. 6 is an example embodiment of a case dashboard 600 for a medical practitioner. The case dashboard 600 enables access to cases owned by the medical practitioner and cases shared with the medical practitioner by other users 155 as indicated in the case summary 610. In this example, the case dashboard 600 is organized as a set of case cards 605 that each graphically show a summary of a case. Selecting a case card 605 links to a case page 400 for the case. In alternative embodiments, the dashboard 600 may be presented in a list view or other view without necessarily presenting case cards 605 in the visual form shown in FIG. 6.

FIG. 7 shows an example of a telepresence interface 700 associated with a telepresence session that may take place during an actual procedure or during a simulated procedure. Alternatively, the telepresence session may be utilized for live planning purposes without necessarily performing or simulating a procedure. In this example, the telepresence interface 700 displays a three-dimensional model of a target anatomy 705 associated with the procedure. The model may include annotated comments that may be obtained during the telepresence session or that were added in a preprocedural stage. Alternatively, the telepresence interface 700 may include a view of real-time video or images associated with an ongoing procedure. In an embodiment, each contributor may be able to switch between different relevant views such as real-time video or images, three-dimensional models, preprocedural images, or other relevant multimedia.

The telepresence interface 700 may furthermore include a telepresence content feed 715 for sending and receiving real-time messages between contributors. For example, a telepresence content feed 715 allows users to post messages and/or view messages from other participants. The messages may include text, media content (e.g., images, video, animations, etc.), or links to various media content or other resources (e.g., research articles). The telepresence interface 700 may furthermore enable participants to provide annotations on the target anatomy (presented in the form of an image, video, or model). For example, a participant may pin a comment to a specific location in the depicted anatomy, as may be indicated by an identifier 710.

Additionally, the telepresence interface 700 may display statistics 720 or other analytics that may be relevant to the procedure. The statistics 720 maybe include estimated or modeled values or metrics relating to the anatomy based on various sensed data from the medical equipment 160. The telepresence interface 700 may dynamically update the statistics 720 over time during the procedure.

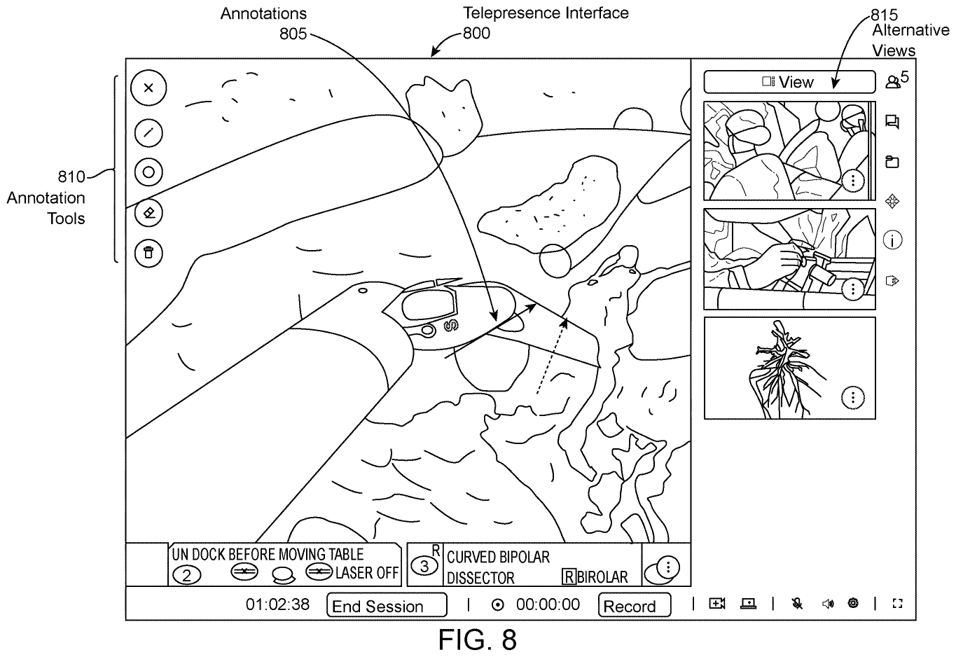

FIG. 8 is another example of a telepresence interface 800 associated with a telepresence session. In this example, the telepresence interface 800 shows a live video of a procedure being performed together with a set of annotation tools 810 that enables a remote contributor to add annotation 805 overlaid on the video. The telepresence interface 800 also includes a set of alternative views 815 the contributor can switch between during the telepresence session. These alternative views 815 may include one or more different camera views (e.g., a view of the medical environment), one or more three-dimensional models (e.g., as shown in FIG. 7), views of preprocedural images, or other multimedia associated with the case.

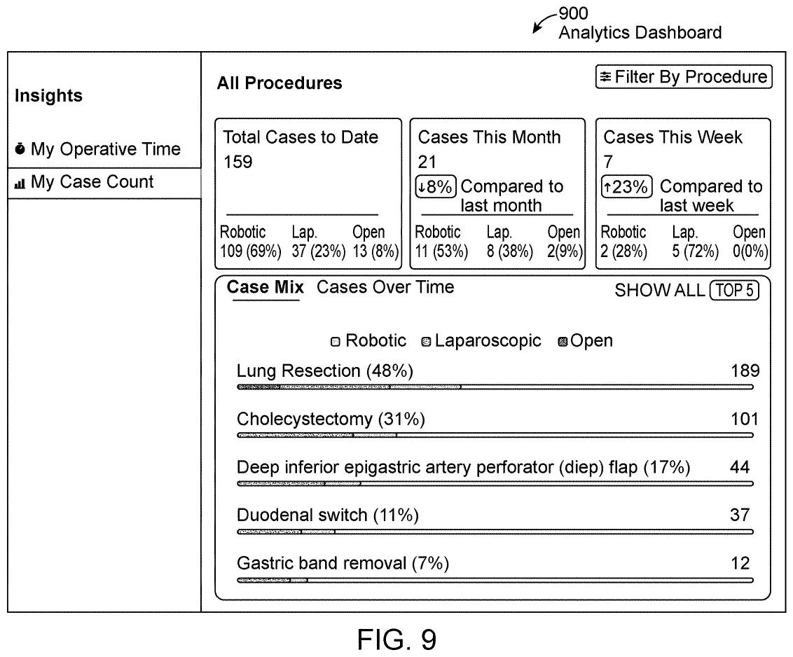

FIG. 9 is an example embodiment of an analytics dashboard 900 for a medical practitioner. In this example, the analytics dashboard 900 displays a summary of cases managed by the medical practitioner includes, for example, a total number of cases, a number of cases in the current month, a number of cases in the current week, and a distribution of types of cases the practitioner has performed.

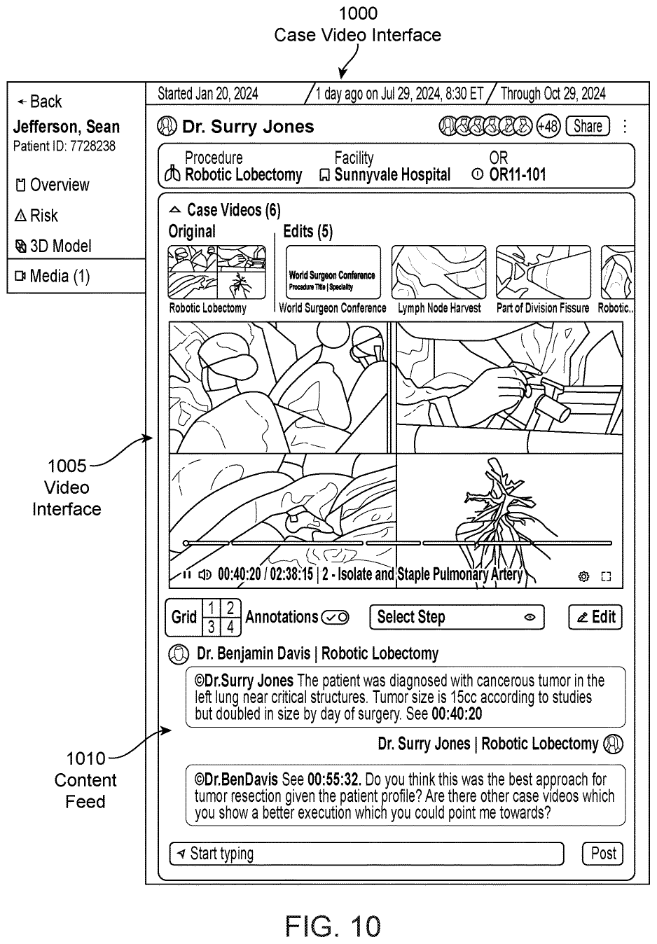

FIG. 10 is an example embodiment of case video interface dashboard 1000 for viewing a case video. Case videos may be captured during a telepresence session or may be similarly captured during a procedure without a live streamed telepresence session. The case video interface 1000 includes a video interface that shows one or more views of a video associated with a medical procedure. The video interface 1000 may include multiple captured views, which may be from cameras in the medical environment, cameras inserted into the anatomy (e.g., endoscopy cameras), or other cameras. Captured views may furthermore include three-dimensional models, preprocedural images, procedure planning documents, or other visual information. The video may be segmented (manually or automatically using video processing and content recognition techniques) to divide the video into segments associated with different steps of the procedure. The video may include annotations provided by a medical practitioner during a telepresence session or in a postprocedural review. A content feed 1010 may be presented in association with a video to enable users 155 to post comments, links, media, or other content in association with the presentation.

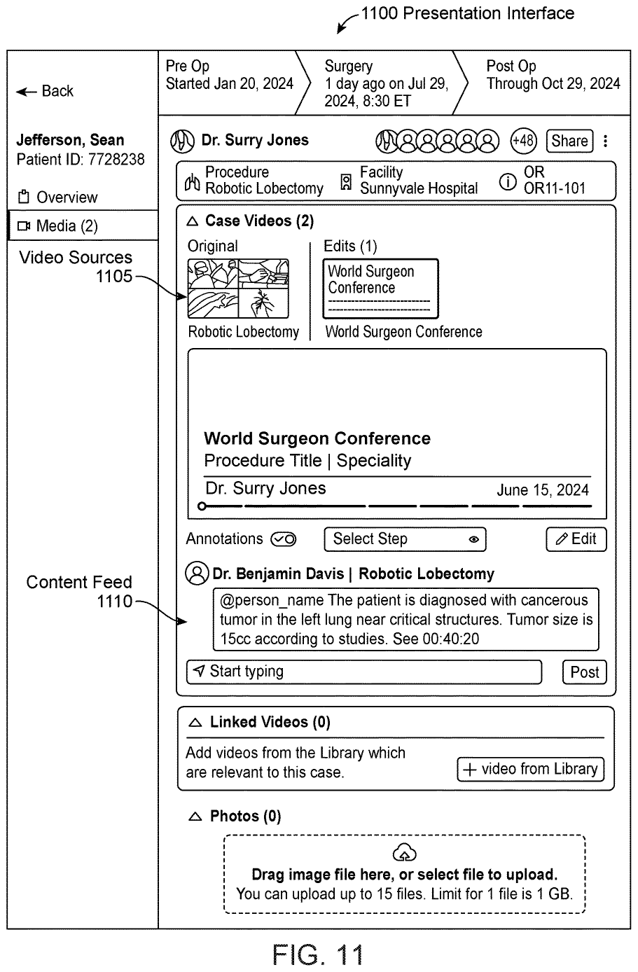

FIG. 11 is an example embodiment of a presentation interface 1100 for creating a presentation from content in the collaborative medical platform 140. In this example, the presentation may be generated from one or more video sources 1105 as a starting point. The video source may then be edited to add contextual slides, commentary, annotations, or other information to create a presentation. In an embodiment, the presentation interface 1100 may enable the presentation to be jointly edited by two or more contributors. The presentation may then be posted on the collaborative medical platform 140 and made accessible to other users 155. In an embodiment, a content feed 1110 may be presented in association with a created presentation to enable users 155 to post comments, links, media, or other content in association with the presentation. A created presentation may be exported or may be case directly from the presentation interface 1110 to an external display.

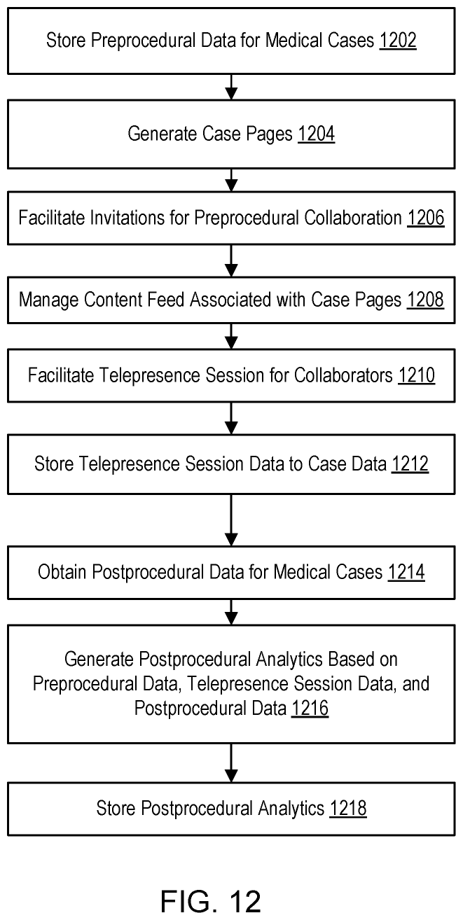

FIG. 12 is an example embodiment of process for operating a collaborative medical platform 140. The collaborative medical platform 140 stores 1202 case data associated with a medical case that includes preprocedural data obtained prior to performance of a medical procedure. The collaborative medical platform 140 generates 1204 a case page for the medical case that enables access to the case data by one or more client devices 150 over the network 130. The collaborative medical platform facilitates 1206 transmission of invitations to a set of contributors to grant access to the case page. For example, the invitations may be created by a case owner that creates an invitation to share the medical case with one or more other practitioners. Furthermore, a machine learning model could be applied to the case data and to profile data for a set of candidate contributors to infer a recommended contributor for inviting to the medical case. The recommended contributor may then be included in the set of invitations.

The collaborative medical platform manages 1208 a content feed associated with the case page that enables the contributors to collaboratively post content to the content feed. For example, contributors may contribute text commentary, multimedia, articles, or links to various content available in the collaborative medical platform 140. In some embodiments, one or more machine learning models may be applied to case data and/or information in the content feed to enable automatic generation of posts for the content feed that may be relevant to the conversation or otherwise relevant to the medical case. For example, in one embodiment, one or more machine learning models may be applied to the case data and to content in the content feed to automatically generate a post including a link to a historical medical case page associated with a historical case that the one or more machine learning models infers is relevant to the medical case.

When the procedure takes place, the collaborative medical platform 140 may facilitate 1210 a telepresence session between the contributors and the medical environment (e.g., operating room) to enable the collaborators to view live video of the procedure and/or access other data such as medical equipment telemetry data, image data, robotic control data, or other data. Furthermore, the telepresence session includes a telepresence interface that includes a content feed for contributors to post commentary or other content during the procedure. The telepresence interface may furthermore include annotations tools that allow remote contributors to annotate video, three-dimensional models, or images associated with the procedure. The telepresence session data may be recorded and stored 1212 to the case data. This may enable the entire telepresence session or portions thereof to be replayed at a later date. Furthermore, the stored data may contribute to various analytics that may be aggregated across medical cases in the collaborative medical platform 140 and may provide various insights for evaluating practitioner performance and/or improving patient outcomes.

The collaborative medical platform 140 furthermore obtains 1214 postprocedural data for the medical case that may relate to patient treatment plans, monitoring of patient outcomes, or other data. This information may be similarly stored in association with the medical case. The collaborative medical platform 140 may generate 1216 various postprocedural analytics based on the procedural data, telepresence session data, and/or the postprocedural data. Analytics may be based on an individual medical case or may be aggregated across multiple medical cases using various filtering parameters and combining functions. The analytics may include various metrics to evaluate practitioner performance, characterize patient outcomes, or provide other medical insights. The postprocedural analytics may be stored 1218 to the collaborative medical platform 140. In an embodiment, one or more machine learning models may be applied to automatically make recommendations based on the insights, such as recommending a training session for a practitioner, recommending a different treatment plan for the patient, etc.

The described embodiments incorporate multiple technical improvements that improve the functioning of computer systems, machine learning techniques, data management systems (particularly as related to healthcare data management), computer-based user interfaces, robotic and/or other medical instrumentation systems, and other technologies and technical fields. For example, the described embodiments provide technical improvements in data availability and data privacy because it enables automated processing of sensitive and/or restricted data such as operating room video (or other video of a medical procedure), patient health records, or other sensitive health data.

The described embodiments furthermore include improvements in machine learning methods in that they combine information from disparate data sources including medical equipment telemetry data, video data, and mobile device data to improve predictive power relative to traditional machine learning techniques. Further still, the described embodiments provide technical improvements in treatment of medical conditions by enabling generation of various notifications, recommendations, or other content tailored to specific medical practitioners that enable them to improve their practice and accordingly results in better patient outcomes.

Furthermore, the described embodiments include technical improvements in the field of robotic-assisted surgery by enabling automated configuration of surgical robots based on the accumulated and aggregated healthcare data associated with patients, facilities, and practitioners. This results in improved performance of the robot, improved human-robot interactions, and improved patient outcomes.

The foregoing description of the embodiments has been presented for the purpose of illustration; it is not intended to be exhaustive or to limit the embodiments to the precise forms disclosed. Persons skilled in the relevant art can appreciate that many modifications and variations are possible in light of the above disclosure.

Some portions of this description describe the embodiments in terms of algorithms and symbolic representations of operations on information. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. Embodiments may also relate to an apparatus for performing the operations herein. This apparatus may be specially constructed for the required purposes, and/or it may include a general-purpose computing device selectively activated or reconfigured by a computer program stored in the computer. Such a computer program may be stored in a tangible non-transitory computer readable storage medium or any type of media suitable for storing electronic instructions and coupled to a computer system bus. Furthermore, any computing systems referred to in the specification may include a single processor or may include architectures employing multiple processor designs for increased computing capability.

Finally, the language used in the specification has been principally selected for readability and instructional purposes, and it may not have been selected to delineate or circumscribe the inventive subject matter. It is therefore intended that the scope is not limited by this detailed description, but rather by any claims that issue on an application based hereon. Accordingly, the disclosure of the embodiments is intended to be illustrative, but not limiting, of the scope of the invention, which is set forth in the following claims.

What is claimed is:

1. A method for operating an online collaborative medical platform, the method comprising:
storing to a database of the online collaborative medical platform, case data associated with a medical case including preprocedural data obtained prior to performance of a medical procedure;
generating a case page for the medical case that enables access to the case data, the case page accessible from the online collaborative medical platform over a network;
facilitating transmission of invitations over the network to enable a set of contributors to gain access to the case page;
managing a content feed associated with the case page that enables the set of contributors to collaboratively post content to the content feed of the case page using respective contributor client devices;
facilitating a telepresence session in a telepresence interface during a medical procedure associated with the medical case, the telepresence interface accessible to the set of contributors over the network via the respective contributor client devices, the telepresence interface including a video stream of the medical procedure and control elements for enabling interaction between the set of contributors;
storing telepresence session data recorded from the telepresence session to the case data associated with the medical case;
obtaining postprocedural data associated with a patient outcome of the medical case;
generating postprocedural analytics based at least in part on the preprocedural data, the telepresence session data, and the postprocedural data for the medical case; and
storing the postprocedural analytics to the database of the online collaborative medical platform.

2. The method of claim 1, wherein facilitating the transmission of the invitations comprises:
receiving a user selection of the set of contributors via a user interface control; and
sending the invitations based on the user selection.

3. The method of claim 1, wherein facilitating the transmission of the invitations comprises:
applying a machine learning model to the case data and to profile data for a set of candidate contributors to infer a recommended contributor for inviting to the medical case; and
including the recommended contributor in the set of invitations.

4. The method of claim 1, wherein managing the content feed comprises:
applying one or more machine learning models to the case data and to content in the content feed to automatically generate a natural language response to a post in the content feed.

5. The method of claim 3, wherein managing the content feed comprises:
applying the one or more machine learning models to the case data and to content in the content feed to automatically generate a post including a link to a historical medical case page associated with a historical case that the one or more machine learning models infers is relevant to the medical case.

6. The method of claim 1, wherein generating the case page comprises:
applying one or more machine learning models to generate a risk assessment for a patient associated with the medical case based in part on patient-specific data and based in part on postprocedural analytics for a set of historical medical cases managed by the online collaborative medical platform.

7. The method of claim 1, wherein generating the postprocedural analytics comprises:
determining a performance metric for a medical practitioner based in part on the case data; and
recommending a training to be completed by the medical practitioner in response to the performance metric failing to meet predefined performance criteria.

8. The method of claim 1, further comprising:
providing user interface tools to facilitate creation of a presentation that incorporates at least one of the preprocedural data the telepresence session data, and the postprocedural data for a medical case; and
storing the presentation in association with the medical case.

9. A non-transitory computer-readable storage medium storing instructions for operating an online collaborative medical platform, the instructions when executed by one or more processors causing the one or more processors to perform steps including:
storing to a database of the online collaborative medical platform, case data associated with a medical case including preprocedural data obtained prior to performance of a medical procedure;
generating a case page for the medical case that enables access to the case data, the case page accessible from the online collaborative medical platform over a network;
facilitating transmission of invitations over the network to enable a set of contributors to gain access to the case page;
managing a content feed associated with the case page that enables the set of contributors to collaboratively post content to the content feed of the case page using respective contributor client devices;
facilitating a telepresence session in a telepresence interface during a medical procedure associated with the medical case, the telepresence interface accessible to the set of contributors over the network via the respective contributor client devices, the telepresence interface including a video stream of the medical procedure and control elements for enabling interaction between the set of contributors;
storing telepresence session data recorded from the telepresence session to the case data associated with the medical case;
obtaining postprocedural data associated with a patient outcome of the medical case;
generating postprocedural analytics based at least in part on the preprocedural data, the telepresence session data, and the postprocedural data for the medical case; and
storing the postprocedural analytics to the database of the online collaborative medical platform.

10. The non-transitory computer-readable storage medium of claim 9, wherein facilitating the transmission of the invitations comprises:
receiving a user selection of the set of contributors via a user interface control; and
sending the invitations based on the user selection.

11. The non-transitory computer-readable storage medium of claim 9, wherein facilitating the transmission of the invitations comprises:
applying a machine learning model to the case data and to profile data for a set of candidate contributors to infer a recommended contributor for inviting to the medical case; and
including the recommended contributor in the set of invitations.

12. The non-transitory computer-readable storage medium of claim 9, wherein managing the content feed comprises:
applying one or more machine learning models to the case data and to content in the content feed to automatically generate a natural language response to a post in the content feed.

13. The non-transitory computer-readable storage medium of claim 12, wherein managing the content feed comprises:
applying the one or more machine learning models to the case data and to content in the content feed to automatically generate a post including a link to a historical medical case page associated with a historical case that the one or more machine learning models infers is relevant to the medical case.

14. The non-transitory computer-readable storage medium of claim 9, wherein generating the case page comprises:
applying one or more machine learning models to generate a risk assessment for a patient associated with the medical case based in part on patient-specific data and based in part on postprocedural analytics for a set of historical medical cases managed by the online collaborative medical platform.

15. The non-transitory computer-readable storage medium of claim 9, wherein generating the postprocedural analytics comprises:
determining a performance metric for a medical practitioner based in part on the case data; and
recommending a training to be completed by the medical practitioner in response to the performance metric failing to meet predefined performance criteria.

16. The non-transitory computer-readable storage medium of claim 9, further comprising:
providing user interface tools to facilitate creation of a presentation that incorporates at least one of the preprocedural data the telepresence session data, and the postprocedural data for a medical case; and
storing the presentation in association with the medical case.

17. A computer system comprising:
one or more processors; and
a non-transitory computer-readable storage medium storing instructions for operating an online collaborative medical platform, the instructions when executed by the one or more processors causing the one or more processors to perform steps including:
storing to a database of the online collaborative medical platform, case data associated with a medical case including preprocedural data obtained prior to performance of a medical procedure;
generating a case page for the medical case that enables access to the case data, the case page accessible from the online collaborative medical platform over a network;
facilitating transmission of invitations over the network to enable a set of contributors to gain access to the case page;
managing a content feed associated with the case page that enables the set of contributors to collaboratively post content to the content feed of the case page using respective contributor client devices;
facilitating a telepresence session in a telepresence interface during a medical procedure associated with the medical case, the telepresence interface accessible to the set of contributors over the network via the respective contributor client devices, the telepresence interface including a video stream of the medical procedure and control elements for enabling interaction between the set of contributors;
storing telepresence session data recorded from the telepresence session to the case data associated with the medical case;
obtaining postprocedural data associated with a patient outcome of the medical case;
generating postprocedural analytics based at least in part on the preprocedural data, the telepresence session data, and the postprocedural data for the medical case; and
storing the postprocedural analytics to the database of the online collaborative medical platform.

18. The computer system of claim 17, wherein facilitating the transmission of the invitations comprises:
receiving a user selection of the set of contributors via a user interface control; and
sending the invitations based on the user selection.

19. The computer system of claim 17, wherein facilitating the transmission of the invitations comprises:
applying a machine learning model to the case data and to profile data for a set of candidate contributors to infer a recommended contributor for inviting to the medical case; and
including the recommended contributor in the set of invitations.

20. The computer system of claim 17, wherein managing the content feed comprises:
applying one or more machine learning models to the case data and to content in the content feed to automatically generate a natural language response to a post in the content feed.
